(19) United States
(12) Patent Application Publication
(10) Pub. No.: US 2024/0115516 A1
(43) Pub. Date: Apr. 11, 2024

(54) TRANSDERMAL DELIVERY OF PRAMIPEXOLE AND RASAGILINE

(71) Applicant: Chin-Chih CHIANG, West Covina, CA (US)

(72) Inventor: Chin-Chih CHIANG, West Covina, CA (US)

(21) Appl. No.: 18/481,850

(22) Filed: Oct. 5, 2023

Related U.S. Application Data
(60) Provisional application No. 63/415,059, filed on Oct. 11, 2022.

(51) Int. Cl. A61K 9/70; A61K 31/137; A61K 31/428

(52) U.S. Cl. CPC A61K 9/7084; A61K 31/137; A61K 31/428

(57) ABSTRACT

A transdermal patch includes a backing film, an adhesive matrix and a release liner. The adhesive matrix contains two active pharmaceutical ingredients, pramipexole and rasagiline. The transdermal patch is to be applied on skins for 7 days to deliver pramipexole and rasagiline for the treatment of Parkinson's disease.

### CROSS REFERENCE TO RELATED APPLICATION

This application claims priority of U.S. Provisional Application No. 63/415,059 filed on Oct. 11, 2022 under 35 U.S.C. § 119(e), the entire contents of which are hereby incorporated by reference.

### FIELD OF THE INVENTION

The present invention relates to a transdermal patch which is composed of a backing film, an adhesive matrix and a release liner. The adhesive matrix contains two active pharmaceutical ingredients, pramipexole and rasagiline. The transdermal patch is to be applied on skins and provides a 7-day sustained release to deliver pramipexole and rasagiline for the treatment of Parkinson's disease, which can maintain less fluctuation of blood concentrations by a once for 7-day application to reduce side effects of both drugs for the treatment of Parkinson's disease patients.

### BACKGROUND

Parkinson's disease is a progressive disorder that is caused by degeneration of nerve cells in the part of the brain called the substantia nigra, which controls movement. These nerve cells die or become impaired, losing the ability to produce an important chemical called dopamine. Studies have shown that symptoms of Parkinson's disease develop in patients with an 80 percent or greater loss of dopamine-producing cells in the substantia nigra. Normally, dopamine operates in a delicate balance with other neurotransmitters to help coordinate the millions of nerve and muscle cells involved in movement. Without enough dopamine, this balance is disrupted, resulting in tremor (trembling in the hands, arms, legs and jaw); rigidity (stiffness of the limbs); slowness of movement; and impaired balance and coordination—the hallmark symptoms of Parkinson's disease.

Pramipexole has the ability to stimulate dopamine receptors in the striatum of the brain in the same way as dopamine, allowing patients to control their movement and mitigate their Parkinson's symptoms. Pramipexole is commercially available as pramipexole hydrochloride which is formulated as tablet dosage form. Pramipexole hydrochloride has been indicated for the treatment of Parkinson's disease and restless legs syndrome.

Rasagiline is a selective, irreversible MAO-B inhibitor indicated for the treatment of idiopathic Parkinson's disease. An enzyme called monoamine oxidase (MAO) breaks down neurotransmitters, dopamine. MAO has two forms, MAO-A and MAO-B. MAO-B is generally believed to break down dopamine. Rasagiline prevents the breakdown of dopamine by irreversibly binding to MAO-B. Dopamine is therefore more available, somewhat compensating for the diminished quantities made in the brains of people with Parkinson's disease. Rasagiline is commercially available as rasagiline mesylate which is formulated as tablet dosage form. Rasagiline mesylate has been indicated for the treatment of idiopathic Parkinson's disease.

The mechanism of action of pramipexole is different from that of rasagiline. The combination of these two drugs may show synergistic effects on the treatment of Parkinson's disease.

The combination of pramipexole and rasagiline aims to demonstrate the potentially synergistic mechanisms is shown in Phase 2 data of a significant symptomatic efficacy of P2B001, an oral tablet, versus placebo, with a benign safety profile that was similar to placebo. Clinical results of an oral administration of the combination once daily show a better efficacy and a lower side effect as if administered individually.

Because of the low molecular weight and its lipophilic property, rasagiline has a fast penetration speed in the patch colloid and will be released from the colloid quickly. There is no rasagiline patch with long-term release in the prior art. The slow release of rasagiline is the problem to be overcome by the present invention, in order to invent a long-term release transdermal patch product including pramipexole and rasagiline, which can be in use of treating Parkinson's disease externally.

### SUMMARY OF THE INVENTION

Methods for solving the above issues are as followings.

1. To construct a double layered patch including a reservoir layer to provide a sufficient amount of rasagiline and a controlling layer to regulate the release of rasagiline (See FIG. 1).

2. To select and add fillers in the controlling layer to allow the release of rasagiline slower and maintain the delivery in a controlled rate for a longer duration.

The present invention develops a 7-day patch by the transdermal delivery of the combination to further increase the patient compliance and maintain less fluctuation of blood concentrations by a once for 7-day application to reduce side effects of both drugs for the treatment of Parkinson's disease patients.

This invention is to design transdermal patch formulations which are composed of two pharmaceutically active ingredients—pramipexole and rasagiline, and other inactive ingredients such as a filler, a plasticizer, a tackifier and a carrier—adhesives and from which pramipexole and rasagiline can permeate through excised human cadaver skins in continuous and stable permeation profiles during 7-day permeation studies.

The transdermal patch is a 3-layered design, including release liner, adhesive matrix and backing layer. The adhesive matrix layer is either a single-layered or a double-layered design.

The single-layered matrix is composed of two pharmaceutically active ingredients and other inactive ingredients which are dissolved in adhesives. The picture of the transdermal patch in the current patent application is shown in FIG. 1.

The double layered matrix is a lamination of two single layered matrix including a reservoir layer and a controlling layer where one side of the reservoir layer is in contact with the backing layer while the other side is laminated with the controlling layer, and where one side of the controlling layer is in contact with the reservoir layer while the other side is in contact with the release liner.

The reservoir layer is enriched with pramipexole and rasagiline to provide sufficient drugs for a 7-day delivery and the controlling layer is designed to control the release of both drugs from the patch and the delivery of drugs across the skins.

Before the application of skin patch, the release liner is removed to expose the adhesive matrix. Upon the application, the active ingredients start releasing from the matrix and are dissolved in the skin and then absorbed by the skin.

The release of active ingredients from the adhesive matrix is by a passive diffusion process. Once the patch is applied on the skin, a concentration gradient is created causing the active ingredients flow through the enriched layer to the less concentrated layer.

If the travel of active ingredients in the adhesive matrix is fast enough to supply those being depleted due to skin absorption during the targeted 7-day application, a single-layered matrix design is designed. If not, a double layered matrix is designed including a reservoir adhesive layer matrix to provide enough active ingredients and a controlling adhesive layer matrix to regulate the release of active ingredients.

### BRIEF DESCRIPTION OF THE DRAWING

FIG. 1 is a schematic view of a single-layered transdermal patch (left), and a double-layered transdermal patch (right).

FIG. 2 is an HPLC Chromatogram for pramipexole (peak 2) and rasagiline (peak 1)

FIG. 3 is skin permeation profile of pramipexole (left) and rasagiline (right) of Examples 1˜5. Example 1—Duro-Tak 387-2052 adhesive having a function group of —COOH. Example 2—Duro-Tak 387-2074 adhesive having function groups of —OH and —COOH. Example 3—Duro-Tak 87-4098 adhesive having no function group. Example 4—Duro-Tak 387-2052 adhesive and Duro-Tak 387-2510 adhesive having a function group of —OH at a ratio of 1:2 by weight. Example 5—Duro-Tak 387-2052 adhesive and Duro-Tak 387-2510 adhesive at a ratio of 2:1 by weight.

FIG. 4 is a skin permeation profile of pramipexole (left) and rasagiline (right): Example 6 is a double-layered adhesive matrix having pramipexole and rasagiline in Duro-Tak 387-2052 and Duro-Tak 387-2510 at 2 to 1 ratio by weight as a reservoir adhesive matrix and Duro-Tak 387-2052 and Duro-Tak 387-2510 at 2 to 1 ratio by weight as the controlling adhesive matrix. Example 7 is a double-layered adhesive matrix having pramipexole and rasagiline in Duro-Tak 387-2052 and Duro-Tak 387-2510 at 1 to 2 ratio by weight as a reservoir adhesive matrix and Duro-Tak 387-2052 and Duro-Tak 387-2510 at 1 to 2 ratio by weight as a controlling adhesive matrix.

FIG. 5 is a skin permeation profile of pramipexole (left) and rasagiline (right): Example 8 is a double-layered adhesive matrix having pramipexole and rasagiline in PSA4302 as a reservoir and PSA4302 as a controlling adhesive matrix. Example 9 is a double-layered adhesive matrix having pramipexole and rasagiline in PSA4602 as a reservoir adhesive matrix and PSA4602 as a controlling adhesive matrix.

FIG. 6 is a skin permeation profile of pramipexole (left) and rasagiline (right): Example 10-Pramipexole and rasagiline in polyisobutylene as a reservoir adhesive matrix and CSD in polyisobutylene as a controlling adhesive matrix. Example 11—Pramipexole and rasagiline in polyisobutylene as a reservoir adhesive matrix and Talc in polyisobutylene as a controlling adhesive matrix. Example 12—Pramipexole and rasagiline in polyisobutylene as a reservoir adhesive matrix and Mg stearate in polyisobutylene as a controlling adhesive matrix. Example 13—Pramipexole and rasagiline in polyisobutylene as a reservoir adhesive matrix and Carbomer 934P in polyisobutylene as a controlling adhesive matrix. Example 14—Pramipexole and rasagiline in polyisobutylene as a reservoir adhesive matrix and Pemulen TR-2 in polyisobutylene as a controlling adhesive matrix.

FIG. 7 is a skin permeation profile of pramipexole (left) and rasagiline (right): Example 15-Pramipexole and rasagiline in polyisobutylene as a reservoir adhesive matrix and Pemulen TR-2 in polyisobutylene as a controlling adhesive matrix. Example 16-Pramipexole and rasagiline in polyisobutylene as a reservoir adhesive matrix and Pemulen TR-2 and CSD in polyisobutylene as a controlling adhesive matrix.

### DETAILED DESCRIPTION OF THE INVENTION

Unless defined otherwise, all technical and scientific terms used herein have the same meanings as is commonly understood by one of skill in the art to which this invention belongs.

As used herein, the articles “a” and “an” refer to one or more than one (i.e., at least one) of the grammatical object of the article. By way of example, “an element” means one element or more than one element.

The term “comprise” or “comprising” is generally used in the sense of include/including which means permitting the presence of one or more features, ingredients or components. The term “comprise” or “comprising” encompasses the term “consists” or “consisting of.”

As used herein, the term “treating” refers to the therapeutic measures to a disease or the symptoms or conditions of a disease, which include but are not limited to applying or administering one or more active agents to a subject suffering from the disease or the symptoms or conditions of the disease or exacerbation of the disease. The purpose of the therapeutic measures is to treat, cure, mitigate, relieve, alter, remedy, ameliorate, improve, or affect the disease, the symptoms or conditions of the disease, disability caused by the disease, or exacerbation of the disease. Specifically, the present invention provides pramipexole and rasagiline for treating Parkinson's disease.

As used herein, the term “effective amount” refers to the amount of an active ingredient achieving desired biological efficacy or therapeutic effects in a subject being treated.

The current invention is related to a transdermal patch product. The patch product is composed of a backing film 1, an adhesive matrix 2, and a release liner 3. The backing film is used as a protective membrane. The adhesive matrix is a lamination of a drug-in-adhesive matrix layer, or a reservoir layer 21, which is containing two pharmaceutically active ingredients, pramipexole and rasagiline and a plasticizer in polyisobutylene adhesive and another adhesive matrix, or a controlling layer 22, which is containing a filler and a plasticizer in polyisobutylene adhesive. The release liner is used as another protective membrane and will be removed before the application of the adhesive matrix to the skin. The backing film is in contact with the reservoir layer and the release liner is in contact with the controlling layer. One patch product is to be applied on skin for 7 days to deliver pramipexole and rasagiline through skin for the treatment of Parkinson's disease.

Ideal drug candidates for the transdermal delivery must be low-molecular weight, lipophilic, low melting point and low daily oral dose. In addition, the transdermal delivery should be more attractive than oral delivery and more patient compliant if its oral bioavailability is low and needs frequent dosings daily. The current application is to describe a transdermal patch for a 7-day application to increase patient compliance. The transdermal patch is a pharmaceutical drug-in-adhesive matrix dosage unit which comprises a backing film, an adhesive matrix, a release liner. Both the backing film and release liner are used as protective membranes. The adhesive matrix is either a single layered drug in adhesive matrix or a double layered adhesive matrix which is a lamination of a reservoir layer, which is composed of two pharmaceutically active ingredients and other inactive ingredients in adhesive matrix and a controlling layer which is an adhesive matrix containing pharmaceutically inactive ingredients. The reservoir layer contains two pharmaceutically active ingredients and other inactive ingredients in adhesive. The controlling layer is a skin contact layer which is composed of inactive ingredients in adhesive which is able to regulate the release of drugs from the reservoir layer. The pharmaceutically active ingredients are pramipexole and rasagiline. The pharmaceutically inactive ingredients are polyisobutylene, an adhesive, a plasticizer, such as mineral oil and a filler, such as Pemulen TR-2, a trade name product of Lubrizol. The in vitro skin permeation studies using human cadaver skins were conducted to evaluate the amounts of drugs delivered through skins.

This invention is to design transdermal patch formulations which are composed of two pharmaceutically active ingredients—pramipexole and rasagiline, and other inactive ingredients such as a filler, a plasticizer, a tackifier and a carrier—adhesives and from which pramipexole and rasagiline can permeate through excised human cadaver skins in continuous and stable permeation profiles during 7-day permeation studies.

The transdermal patch is a 3-layered design, including release liner, adhesive matrix and backing layer. The adhesive matrix layer is either a single-layered or a double-layered design.

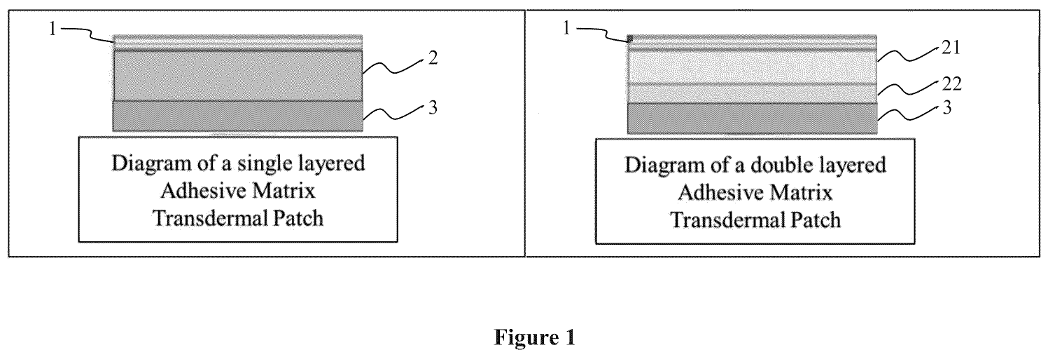

The single-layered matrix is composed of two pharmaceutically active ingredients and other inactive ingredients which are dissolved in adhesives. The picture of the transdermal patch in the current patent application is drawn as shown in FIG. 1.

The double layered matrix is a lamination of two single layered matrix including a reservoir layer and a controlling layer where one side of the reservoir layer is in contact with the backing layer while the other side is laminated with the controlling layer, and where one side of the controlling layer is in contact with the reservoir layer while the other side is in contact with the release liner.

The picture of the transdermal patch in the current patent application is drawn and shown in FIG. 1.

The reservoir layer is enriched with pramipexole and rasagiline to provide sufficient drugs for a 7-day delivery and the controlling layer is designed to control the release of both drugs from the patch and the delivery of drugs across the skins.

Before the application of skin patch, the release liner is removed to expose the adhesive matrix. Upon the application, the active ingredients start releasing from the matrix and are dissolved in the skin and then absorbed by the skin.

The release of active ingredients from the adhesive matrix is by a passive diffusion process. Once the patch is applied on the skin, a concentration gradient is created causing the active ingredients flow through the enriched layer to the less concentrated layer.

If the travel of active ingredients in the adhesive matrix is fast enough to supply those being depleted due to skin absorption during the targeted 7-day application, a single-layered matrix design is designed. If not, a double layered matrix is designed including a reservoir adhesive layer matrix to provide enough active ingredients and a controlling adhesive layer matrix to regulate the release of active ingredients.

A present invention is a transdermal patch formulation having pharmaceutically active ingredients of pramipexole and rasagiline and other inactive ingredients of a filler, a plasticizer and a tackifier in an adhesive, the carrier.

A further object of the present invention is the above formulation in which pramipexole is in its base form and having its chemical name of (S)-2-amino-4,5,6,7-tetrahydro-6-(propylamino)benzothiazole, its empirical formula of C₁₀H₁₇N₃S and a molecular weight of 211.3 and in which rasagiline is in its base form and having its chemical name of (R)—N-(2-Propynyl)-2,3-dihydroinden-1-amine, its empirical formula of C₁₂H₁₃N and a molecular weight of 171.2.

In accordance of the present invention, the carrier is an adhesive in which two adhesive-matrix are formed, i.e., a reservoir layer containing drugs and a plasticizer in an adhesive which is designed to provide sufficient amounts of drugs, and a controlling layer containing a filler in an adhesive which is designed to regulate the drug release from the transdermal patch.

In accordance of the present invention, the adhesive in the above formulation is selected from the group consisting of polyacrylate adhesive, silicon adhesive and polyisobutylene adhesive.

In accordance of the present invention, the most preferred adhesive is polyisobutylene. Polyisobutylene adhesives are marketed by BASF (sold under the names of Oppanol N-100, a high viscosity polymer having an average molecular weight of about 1,000,000 and Oppanol B-10, a low viscosity polymer having an average molecular weight of 10,000-50,000). A combination of Oppanol N-100 and Oppanol B-10 at a ratio of 1.0:1.5 to 1.5:1.0 by weight is preferred.

A further object of the present invention is the above formulation in which the filler is selected from the group consisting of colloidal silicon dioxide, acrylic acid polymers (sold under the name of Carbomer 934P), polyvinyl pyrrolidone K90, and Acrylates/C10-30 Alkyl Acrylate Crosspolymer (sold under the names of Pemulen TR-1, or Pemulen TR-2).

The most preferred filler is Pemulen TR-1 or Pemulen TR-2. Pemulen TR-2 is the trade name product of Lubrizol. Pemulen TR-2 is a polymeric emulsifier of a high molecular weight, crosslinked copolymer of acrylic acid and a hydrophobic C10-30 alkyl acrylate co-monomer. Its INCI Name is Acrylates/C10-30 Alkyl Acrylate Crosspolymer.

In accordance of the present invention, the amount of the filler is formulated in the range of 0.1% to 10% by weight.

A further object of the present invention is the above formulation in which a plasticizer is selected from the group consisting of mineral oil, light and mineral oil, heavy.

The most preferred plasticizer is mineral oil. In accordance of the present invention, the amount of the plasticizer is used in the range of 10% to 30% by weight.

A further object of the present invention is the above formulation in which the tackifier is selected from the group consisting of colloidal silicon dioxide, talc and magnesium stearate. A tackifier is used to adjust the integrity of the polyisobutylene based adhesive matrix.

The most preferred tackifier is colloidal silicon dioxide. In accordance of the present invention, the amount of the tackifier is formulated in the range of 5% to 30% by weight.

### EXAMPLES

### Examples 1-5 (Single-Layered Polyacrylate Adhesive Matrix)

The combination of pramipexole and rasagiline were formulated into polyacrylate adhesives, including those adhesives having different functional groups such as Duro-Tak 387-2052, Duro-Tak 87-2074, Duro-Tak 87-4908 and Duro-Tak 387-2516, which are having —OH/—COOH, none and —OH functional groups in their polyacrylate structures, respectively. Formulations of Example 1 to Example 5 are shown in the following table (Table 1). Patches which have a single-layered matrix are produced.

The formulations are pramipexole and rasagiline dissolved in Duro-Tak solutions. These solutions contain evaporable solvents, such as ethyl acetate and/or isopropyl alcohol which are removed during patch manufacturing process and the dried adhesive films are to be formed. Each dried film is designed to contain 8% pramipexole and 2% rasagiline.

Example 1—Duro-Tak 387-2052 adhesive having a function group of —COOH. Example 2—Duro-Tak 387-2074 adhesive having function groups of —OH and —COOH. Example 3—Duro-Tak 87-4098 adhesive having no function group. Example 4—Duro-Tak 387-2052 adhesive and Duro-Tak 387-2510 adhesive having a function group of —OH at a ratio of 1:2 by weight. Example 5—Duro-Tak 387-2052 adhesive and Duro-Tak 387-2510 adhesive at a ratio of 2:1 by weight.

TABLE 1
Formulations of Examples 1-5

| | | Example | | | | |
|---|---|---|---|---|---|---|
| | Function Groups of | 1 | 2 | 3 | 4 | 5 |
| Ingredients | Adhesives | Weight % | | | | |
| Pramipexole | — | 8 | 8 | 8 | 8 | 8 |
| Rasagiline | — | 2 | 2 | 2 | 2 | 2 |
| Lauroglycol* | — | 2 | 2 | 2 | 2 | 2 |
| Duro-Tak 387-2052 | —COOH | 88 | — | — | — | — |
| Duro-Tak 387-2074 | ′—OH/COOH | — | 88 | — | — | — |
| Duro-Tak 87-4098 | None | — | — | 88 | — | — |
| Duro-Tak 387-2052 & | —COOH and —OH | — | — | — | 88 | — |
| Duro-Tak 387-2510 | | | | | | |
| (1:2 by weight) | | | | | | |
| Duro-Tak 387-2052 & | —COOH and —OH | — | — | — | — | 88 |
| Duro-Tak 387-2510 | | | | | | |
| (2:1 by weight) | | | | | | |

Each ingredient is listed as the percentage weight.
*The chemical name of Lauroglycol is Propylene glycol monolaurate. It is a trade name sold by Gattefosse. It is commonly used as a skin permeation enhancer.

Manufacture of patches of Example 1 is described as in the following. Manufacture of other patches (Examples 2-5) follows the same process.

Add and dissolve 1.0 gm pramipexole and 0.25 gm rasagiline in a beaker containing 51.4 gm Duro-Tak 387-2052. Stir until homogeneous. Fix a sheet of release liner on the tray of Warner Mathis Coater. Pour approximately 5 gm of the drug containing adhesive solution on to the release liner. Use doctor knife to coat the adhesive solution evenly at a controlled thickness of 0.4 mm on the release liner. Send the tray into the drying oven which is preset at 60° C. for a drying time of 10 minutes. After drying, the tray is released out of the oven. Cover the dried drug containing adhesive film with a sheet of backing layer. Release the 3-layer sheet from the coating frame and cut the sheet into patches of desired sizes using a carver press die cutter.

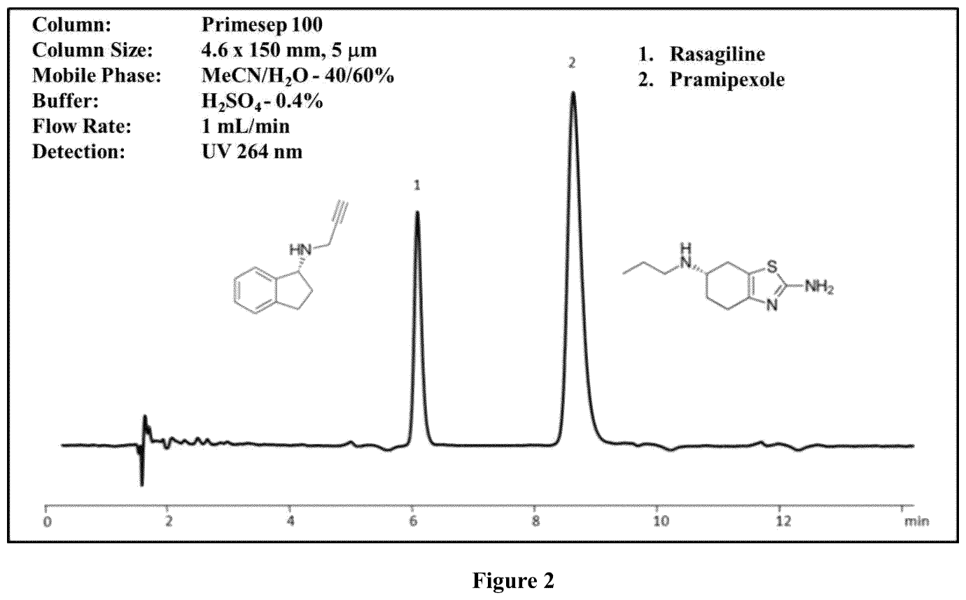

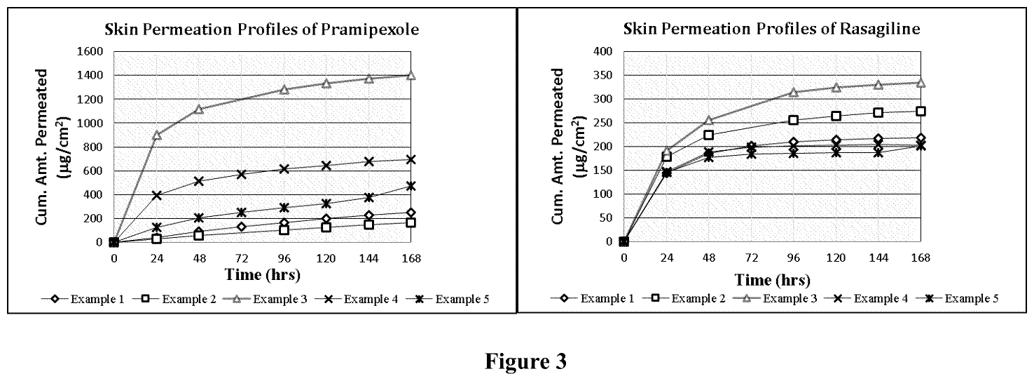

The patches thus made are subjected to the in-vitro skin permeation studies. A horizontal type skin permeation set up is used. Human cadaver skins are used as membrane having a calculated active permeation area of 0.64 cm². Each patch is applied on to the stratum corneum site of skin and the dermal site of skin is attached facing to each receptor compartment. In each receptor compartment, 3.5 mL of 20% polyethylene glycol 400 is added and used as the receptor medium. Samples, 0.1 mL each are taken with replacement every 24 hours to up to 168 hours. All samples were quantitated for pramipexole and rasagiline concentrations by HPLC. A typical HPLC chromatogram is shown in FIG. 2. Cumulated amount of pramipexole and rasagiline (in μg/cm²) versus time (in hours) profiles are plotted in FIG. 3 for Examples 1-5.

It is shown on FIG. 3 that pramipexole in Example 3 which is using the adhesive with no functional groups shows the highest skin permeation while that in the Example 1 which is using the adhesive with —COOH shows the lowest skin permeation profile. There is no continuous and stable absorption for Example 3, however, there is a 7-day continuous and stable absorption for Example 1. Surprisingly, as for rasagiline, all absorptions are showing non-linear profiles.

TABLE 2
The amount permeated across the skins from Example 3

| | Amt. permeated (μg/cm²/hr) | | | | | | |
|---|---|---|---|---|---|---|---|
| Example 1 | Day 1 | Day 2 | Day 3 | Day 4 | Day 5 | Day 6 | Day 7 |
| Pramipexole | 1.52 | 1.85 | 1.82 | 1.71 | 1.64 | 1.56 | 1.48 |
| Rasagiline | 5.99 | 3.87 | 2.80 | 2.18 | 1.78 | 1.50 | 1.30 |

A constant delivery is averaged at 1.65 μg/cm²/hr during the 7-day delivery for pramipexole. However, it is not the case for rasagiline. The amount delivered is 5.99 μg/cm²/hr and decreases throughout other days.

### Examples 6-7 (Double-Layered Polyacrylate Adhesive Matrix)

Formulations of Example 6-7 are shown in the following table (Table 3). Example 6-Double-layered adhesive matrix having pramipexole and rasagiline in Duro-Tak 387-2052 and Duro-Tak 387-2510 at 2 to 1 ratio by weight as a reservoir adhesive matrix and Duro-Tak 387-2052 and Duro-Tak 387-2510 at 2 to 1 ratio by weight as the controlling adhesive matrix. Example 7—Double-layered adhesive matrix having pramipexole and rasagiline in Duro-Tak 387-2052 and Duro-Tak 387-2510 at 1 to 2 ratio by weight as a reservoir adhesive matrix and Duro-Tak 387-2052 and Duro-Tak 387-2510 at 1 to 2 ratio by weight as a controlling adhesive matrix.

TABLE 3
Formulations of Example 6-7

| | | Example | |
|---|---|---|---|
| | | 6 | 7 |
| Layer | Ingredients | Weight % | |
| Reservoir | Pramipexole | 8 | 8 |
| | Rasagiline | 1 | 1 |
| | Duro-Tak 387-2052 | 30 | 61 |
| | Duro-Tak 387-2510 | 61 | 30 |
| Controlling | Duro-Tak 387-2052 | 53.3 | 52 |
| | Duro-Tak 387-2510 | 26.7 | 26 |
| | PVP K90 | 20 | 20 |
| | SAIB | - - - | 2 |

Each ingredient is listed as the percentage weight.

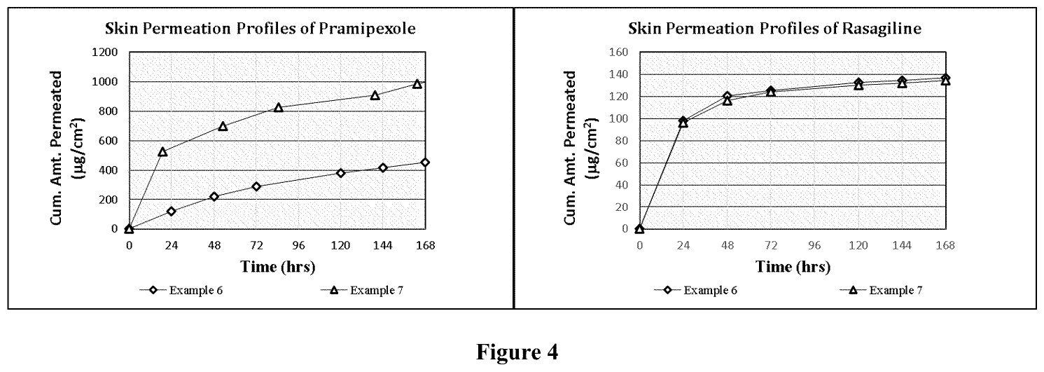

Skin permeation studies were performed. As shown in FIG. 4, the skin permeation of pramipexole slows down with the controlling layer having the polyacrylate adhesives and PVP only. Pramipexole shows a higher skin permeation when formulated in the adhesive with 2% SAIB (Sucrose Acetate Isobutyrate) added. For rasagiline, its skin permeation profiles show fast skin permeations on day 1 and slow down afterwards. In addition, the permeation is not different if additives in the controlling layer are different.

### Examples 8-9 (Double-Layered Silicone Adhesive Matrix)

Manufacture of patches of Example 8 is described as in the following. Manufacture of patches of other formulations (Example 9) follows the same process.

Transdermal patches are formulated using pressure sensitive adhesives (PSA) as carriers. Two types of PSA, PSA-4302 and PSA-4602 are used. Formulations of Example 8-9 are shown in the following table (Table 4). Example 8 is a double-layered adhesive matrix having pramipexole and rasagiline in PSA4302 as a reservoir and PSA4302 as a controlling adhesive matrix. Example 9 is a double-layered adhesive matrix having pramipexole and rasagiline in PSA4602 as a reservoir adhesive matrix and PSA4602 as a controlling adhesive matrix.

TABLE 4
Formulations of Example 8-9

| | | Example | |
|---|---|---|---|
| | | 8 | 9 |
| Layer | Ingredients | Weight % | |
| Reservoir | Pramipexole | 5 | 5 |
| | Rasagiline | 1 | 1 |
| | PSA4302 | 94 | - - - |
| | PSA4602 | - - - | 94 |
| Controlling | PSA4302 | 100 | - - - |
| | PSA4602 | - - - | 100 |

Each ingredient is listed as the percentage weight.

Manufacturing of the Reservoir Layer: Add and dissolve 1.54 gm pramipexole and 0.31 gm rasagiline in a beaker containing 48.16 gm PSA4302. Stir until homogeneous. Fix a sheet of backing layer on the tray of Warner Mathis Coater. Pour approximately 10 gm of the drug containing adhesive solution on to the backing layer. Use a doctor knife to coat the adhesive solution evenly at a controlled thickness of 0.4 mm on the backing layer. Send the tray into the drying oven which is preset at 60° C. for a drying time of 10 minutes. After drying, the tray is released out of the oven. Release the film and put it aside.

Manufacturing of the Controlling Layer: Fix a sheet of release liner on the tray of Warner Mathis Coater. Pour approximately 10 gm of PSA4302 on to the release liner. Use a doctor knife to coat PSA4302 solution evenly at a controlled thickness of 0.1 mm on the release liner. Send the tray into the drying oven which is preset at 60° C. for a drying time of 10 minutes. After drying, the tray is released out of the oven. Carefully laminate the adhesive side of Reservoir Layer on top the adhesive side of Controlling Layer avoiding air bubbles in between. Release the laminate sheet from the coating frame and cut the sheet into patches of desired sizes using a carver press die cutter.

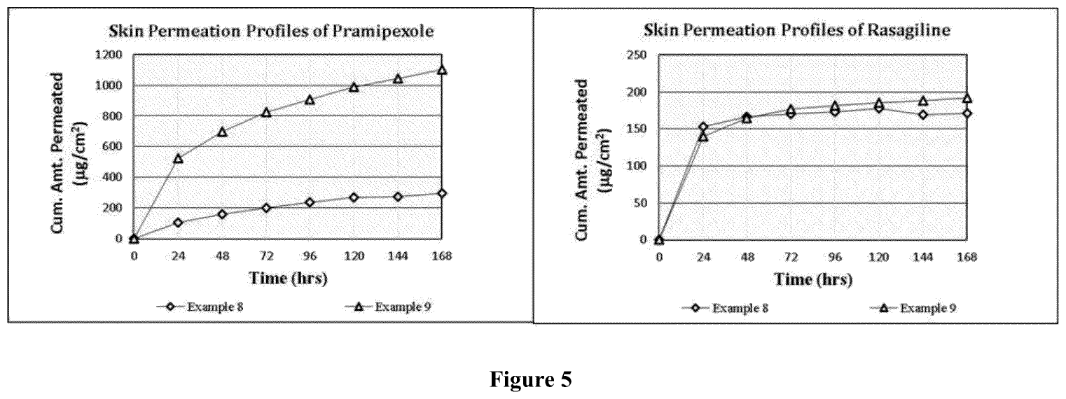

Skin permeation studies were performed. As shown in FIG. 5, pramipexole when formulated in PSA 4602 shows a higher skin permeation than it in PSA 4302. For rasagiline, its skin permeation is the same when formulated in either PSA 4302 or PSA 4602. However, non-linear skin permeations are resulted.

### Examples 10-14 (Double-Layered Polyisobutylene Adhesive Matrix)

Formulations of Example 10-14 are shown in the following table (Table 5). Manufacture of patches of Example 10 is described as in the following. Manufacture of patches of other formulations (Examples 11-14) follows the same process. Example 10-Pramipexole and rasagiline in polyisobutylene as a reservoir adhesive matrix and CSD in polyisobutylene as a controlling adhesive matrix. Example 11—Pramipexole and rasagiline in polyisobutylene as a reservoir adhesive matrix and Talc in polyisobutylene as a controlling adhesive matrix. Example 12—Pramipexole and rasagiline in polyisobutylene as a reservoir adhesive matrix and Mg stearate in polyisobutylene as a controlling adhesive matrix. Example 13—Pramipexole and rasagiline in polyisobutylene as a reservoir adhesive matrix and Carbomer 934P in polyisobutylene as a controlling adhesive matrix. Example 14-Pramipexole and rasagiline in polyisobutylene as a reservoir adhesive matrix and Pemulen TR-2 in polyisobutylene as a controlling adhesive matrix.

TABLE 5
Formulations of Example 10-14

| | | Example | | | | |
|---|---|---|---|---|---|---|
| Layer | Ingredients | 10 | 11 | 12 | 13 | 14 |
| Reservoir | Pramipexole | 5.75 | 5.75 | 5.75 | 5.75 | 5.75 |
| | Rasagiline | 1.44 | 1.44 | 1.44 | 1.44 | 1.44 |
| | Oppanol N-100 | 25.01 | 25.01 | 25.01 | 25.01 | 25.01 |
| | Oppanol B-10 | 31.26 | 31.26 | 31.26 | 31.26 | 31.26 |
| | Mineral Oil, Heavy | 36.55 | 36.55 | 36.55 | 36.55 | 36.55 |
| Controlling | Oppanol N-100 | 24.32 | 24.32 | 24.32 | 25.59 | 25.59 |
| | Oppanol B-10 | 30.4 | 30.4 | 30.4 | 31.99 | 31.99 |
| | Mineral Oil, Heavy | 35.54 | 35.54 | 35.54 | 37.4 | 37.4 |
| | CSD | 9.74 | — | — | — | — |
| | Talc | — | 9.74 | — | — | — |
| | Mg Stearate | — | — | 9.74 | — | — |
| | Carbomer 934P | — | — | — | 5.02 | — |
| | Pemulen TR-2 | — | — | — | — | 5.02 |

Each ingredient is listed as the percentage weight.

Manufacturing of the Reservoir Layer: Add and dissolve Oppanol N-100, Oppanol B-10 and Mineral Oil, heavy in n-heptane to form an adhesive solution. Add pramipexole and rasagiline into the adhesive solution. Homogenize the mixture. Fix a sheet of backing layer on the tray of Warner Mathis Coater. Pour approximately 10 gm of the drug containing adhesive solution on to the backing layer. Use a doctor knife to coat the adhesive solution evenly at a controlled thickness of 0.4 mm on the backing layer. Send the tray into the drying oven which is preset at 60° C. for a drying time of 10 minutes. After drying, the tray is released out of the oven. Release the film and put it aside.

Manufacturing of the Controlling Layer: Add and dissolve Oppanol N-100, Oppanol B-10 and Mineral Oil, heavy in n-heptane to form an adhesive solution. Homogenize Talc in n-heptane. Add the mixture into the adhesive solution and homogenize. Fix a sheet of release liner on the tray of Warner Mathis Coater. Pour approximately 10 gm of the mixture on to the release liner. Use a doctor knife to coat the mixture evenly at a controlled thickness of 0.1 mm on the release liner. Send the tray into the drying oven which is preset at 60° C. for a drying time of 10 minutes. After drying, the tray is released out of the oven. Carefully laminate the adhesive side of Reservoir Layer on the adhesive side of Controlling Layer avoiding air bubbles in between. Release the laminate sheet from the coating frame and cut the sheet into patches of desired sizes using a carver press die cutter.

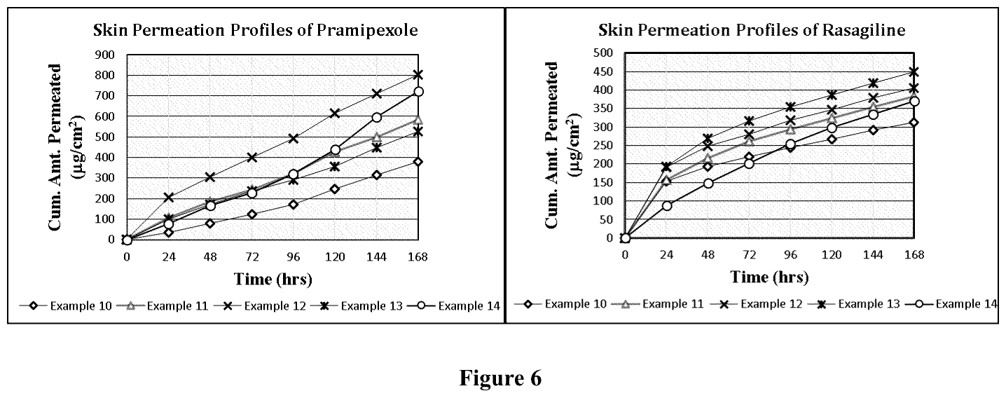

It is shown on FIG. 6 and Table 6 that pramipexole in Example 14 shows a controlled and stable skin delivery for a 7 day duration having an average hourly delivery of 3.62 μg/cm². Surprisingly, rasagiline is also showing a controlled and stable skin delivery for a 7 day duration having an average hourly delivery of 2.75 μg/cm². All other absorptions are showing non-linear profiles.

TABLE 6
The amount permeated across the skins from Example 14

| | Amount Permeated (μg/cm²/hr) | | | | | | |
|---|---|---|---|---|---|---|---|
| Example 14 | Day 1 | Day 2 | Day 3 | Day 4 | Day 5 | Day 6 | Day 7 |
| Pramipexole | 3.28 | 3.43 | 3.17 | 3.32 | 3.67 | 4.14 | 4.30 |
| Rasagiline | 3.68 | 3.11 | 2.81 | 2.66 | 2.49 | 2.33 | 2.20 |

### Examples 15-16 (Double-layered Polyisobutylene Adhesive Matrix)

Formulations of Example 15-16 are shown in the following table (Table 7). Example 15—Pramipexole and rasagiline in polyisobutylene as a reservoir adhesive matrix and Pemulen TR-2 in polyisobutylene as a controlling adhesive matrix. Example 16—Pramipexole and rasagiline in polyisobutylene as a reservoir adhesive matrix and Pemulen TR-2 and CSD in polyisobutylene as a controlling adhesive matrix.

TABLE 7
Formulations of Example 15-16

| | | Example | |
|---|---|---|---|
| | | 15 | 16 |
| Layer | Ingredients | Weight % | |
| Reservoir | Pramipexole | 5.59 | 5.59 |
| | Rasagiline | 1.4 | 1.4 |
| | Oppanol N-100 | 25.06 | 25.06 |
| | Oppanol B-10 | 31.33 | 31.33 |
| | Mineral Oil, Heavy | 36.63 | 36.63 |
| Controlling | Oppanol N-100 | 25.62 | 24.19 |
| | Oppanol B-10 | 32.02 | 30.23 |
| | Mineral Oil, Heavy | 37.44 | 35.35 |
| | Pemulen TR-2 | 4.93 | 5.12 |
| | CSD | - - - | 5.12 |

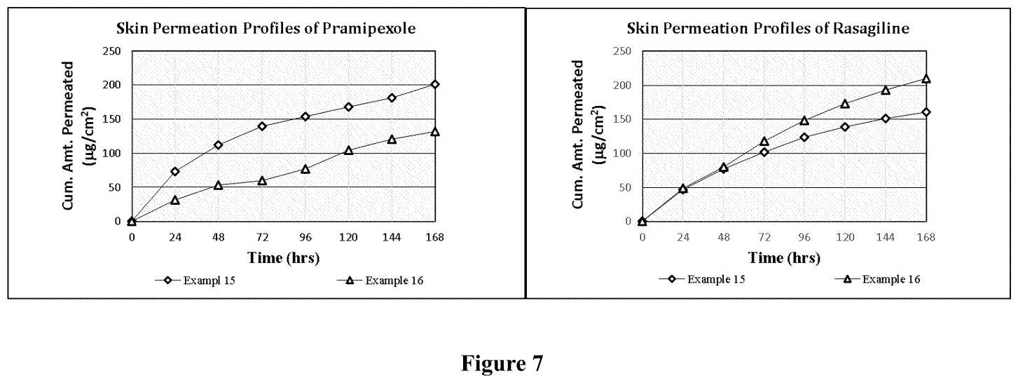

The examples are focused on the formulations of the controlling layer. FIG. 7 shows the skin permeation profile of pramipexole and rasagiline. Example 15 with Pemulen TR-2 in the controlling layer is showing a faster delivery pramipexole than Example 16 which not only has Pemulen TR-2 also has colloidal silicon dioxide (CSD) added. However, CSD seems not much interfering the skin permeation of rasagiline.

Based on the above results, the patch of the present invention can release pramipexole and rasagiline continuously for 7 days. So far there is no patch product containing both pramipexole and rasagiline. It is difficult for pramipexole and rasagiline to be released stably for a long time from the patch. Especially in the prior art, there is no patch product that can release rasagiline for a long time. Because of its small molecular weight and lipophilicity, rasagiline is released from the patch very quickly.

The present invention develops a double-layered adhesive matrix. Patch of the present invention can release pramipexole and rasagiline continuously in a controlled rate for 7 days. The adhesive matrix includes a reservoir layer with active pharmaceutical ingredient and a controlling layer. The reservoir layer includes pramipexole and rasagiline, and is covered with the controlling layer containing water-based polymer carbomer or Acrylates/C10-30 Alkyl Acrylate Crosspolymer, which can absorb oily ingredients, so that rasagiline is first adsorbed and released when it passes through the controlling layer, thereby controlling the release speed of the drug from the patch. Instead of rapidly releasing most of the drug on the first day, an effective amount of the drug can be released steadily every day. The release can last 7 days. This unique formulation design is different from those of patches sold today.

Accordingly, it is to be understood that the embodiments of the invention herein described are merely illustrative of the application of the principles of the invention. Reference herein to details of the illustrated embodiments is not intended to limit the scope of the claims, which themselves recite those features regarded as essential to the invention.

1. A transdermal patch comprising,
a backing layer,
a polymer matrix layer, wherein the polymer matrix is a lamination of two matrices containing a reservoir layer and a controlling layer, wherein the reservoir layer contains a pharmaceutically active ingredient, a first filler, a first plasticizer, and a first tackifier in polyisobutylene adhesive, the pharmaceutically active ingredient is selected from the group consisting of pramipexole and rasagiline, the first filler is selected from the group consisting of colloidal silicon dioxide, acrylic acid polymers, polyvinyl pyrrolidone K90, and Acrylates/C10-30 Alkyl Acrylate Crosspolymer, and wherein the controlling layer contains a second filler, and a second tackifier in polyisobutylene adhesive, the second filler is selected from the group consisting of colloidal silicon dioxide, acrylic acid polymers, polyvinyl pyrrolidone K90, and Acrylates/C10-30 Alkyl Acrylate Crosspolymer; and
a release liner,
wherein the polymer matrix layer is disposed between the backing layer and the release liner, and the pharmaceutically active ingredient is released from the transdermal patch and passed across skins to exert pharmacological activities.

2. The transdermal patch according to claim 1, the first plasticizer is mineral oil.

3. The transdermal patch according to claim 1, the first tackifier is selected from the group consisting of colloidal silicon dioxide, talc and magnesium stearate.

4. The transdermal patch according to claim 1, wherein the controlling layer further contains a second plasticizer.

5. The transdermal patch according to claim 1, the second tackifier is selected from the group consisting of colloidal silicon dioxide, talc and magnesium stearate.

6. The transdermal patch according to claim 4, the second plasticizer is mineral oil.

7. The transdermal patch according to claim 1, wherein the transdermal patch is to be used for a treatment of Parkinson's disease.

8. The transdermal patch according to claim 1, wherein the transdermal patch is to deliver pramipexole at a skin permeation rate of 3-40 mg/cm² per hour.

9. The transdermal patch according to claim 1, wherein the transdermal patch is to deliver rasagiline at a skin permeation rate of 4-10 mg/cm² per hour.

10. The transdermal patch according to claim 1, wherein the transdermal patch is to be used for a 7-day application.
